(19) United States
(12) Patent Application Publication
(10) Pub. No.: US 2017/0360974 A1
(43) Pub. Date: Dec. 21, 2017

(54) Plasmaclave Device

(71) Applicant: Medident Technologies Inc., Surrey (CA)

(72) Inventor: Gilbert Fregoso, Fallbrook, CA (US)

(73) Assignee: Medident Technologies Inc., Surrey (CA)

(21) Appl. No.: 15/628,289

(22) Filed: Jun. 20, 2017

Related U.S. Application Data
(60) Provisional application No. 62/352,971, filed on Jun. 21, 2016.

(51) Int. Cl. A61L 2/14; A61L 2/03; A61L 2/24

(52) U.S. Cl. CPC A61L 2/14; A61L 2/24; A61L 2/03; A61L 2202/24; A61L 2202/14

(57) ABSTRACT

Example plasmaclave devices are shown for using a plasma to sterilize objects or equipment (e.g., medical instruments, surgical devices, food preparation utensils, etc.) placed inside the plasmaclave devices. In some embodiments, the plasmaclave device includes fixed magnets, steering coils, and a high voltage electrode for generating a plasma field and controlling where the plasma field is focused, so that the plasma field is rapidly scanned over a target surface. In some embodiments, the plasmaclave device includes a high voltage coil, a motor, and a blade electrode for generating a plasma field and spinning the blade electrode, so that the plasma field is rapidly scanned over a target surface.

### RELATED APPLICATIONS

This application claims the benefit of U.S. Provisional Patent Application No. 62/352,971 filed on Jun. 21, 2016 and entitled “Plasmaclave Device,” which is hereby incorporated by reference for all purposes.

### BACKGROUND

Reusable medical equipment must be sterilized after each use and before the next use. A common sterilization technique includes autoclaving the medical equipment. In this technique, the medical equipment is placed inside an autoclave device (much like an oven) and heated at a sufficiently high temperature for a sufficiently long period of time to ensure that any pathogens on the medical equipment is killed, destroyed or eliminated. The high temperatures required for such techniques require that the equipment cool down after the process is complete, which adds time to the process. The high temperatures also present a potential hazard to whomever handles the medical equipment after the autoclaving process is completed.

Atmospheric pressure plasma processes for sterilizing objects are known. In these processes, a plasma torch is used to generate a plasma and project the plasma out of an orifice, typically using gas flow. The objects to be sterilized are placed beneath the projected plasma. These atmospheric pressure systems typically focus the plasma to a small spot or line, and in some cases the objects to be sterilized are moved beneath the plasma to cover a larger area.

### SUMMARY

In accordance with some embodiments, a plasmaclave device used to sterilize objects includes a chamber, a stationary tray, a constrained plasma zone, one or more high voltage electrodes, fixed magnets, and steering coils. The stationary tray is disposed within the chamber. The stationary tray has a target surface and is configured to hold the objects on the target surface. The constrained plasma zone is within the chamber adjacent to the target surface of the stationary tray. The high voltage electrodes, the fixed magnets, and the steering coils are disposed within the chamber. The high voltage electrodes generate an electric field that creates a plasma within the chamber. The fixed magnets focus the electric field constraining the plasma into the constrained plasma zone of the of the chamber. The steering coils further constrain the plasma, and scan the further constrained plasma over the target surface of the stationary tray.

In accordance with some embodiments, a method of sterilizing objects includes providing a chamber having a stationary tray and a constrained plasma zone therein, the objects being on a target surface of the stationary tray; creating a plasma within the chamber by generating an electric field therein; pulsing the plasma at a repetition frequency using a power supply; constraining the plasma into the constrained plasma zone by focusing the electric field using fixed magnets; and further constraining the plasma and scanning the further constrained plasma across the objects on the target surface of the stationary tray using steering coils.

In some embodiments, the plasma is a DC plasma. In some embodiments, the plasma is an AC plasma with a frequency from 1 kHz to 1 MHz. In some embodiments, the plasma is pulsed with a repetition frequency from 1 Hz to 1 kHz. In some embodiments, a pressure in the chamber is from 1 Torr to 10 Torr. In some embodiments, a power supply is included that produces the plasma at a power level from 10 W to 100 W per 20 square inches of chamber volume. In some embodiments, the stationary tray comprises aluminum. In some embodiments, a maximum temperature of the objects or equipment being sterilized is less than or equal to 200° C. In some embodiments, the constrained plasma is scanned over the constrained plasma zone in a serpentine pattern. In some embodiments, the constrained plasma is scanned over the constrained plasma zone in a raster pattern. In some embodiments, a time period for completing a scan cycle is between 0.1 second and 1 second per cycle. In some embodiments, the objects or equipment to be sterilized are located in a portion of the constrained plasma zone, and the further constrained plasma is scanned over the portion of the constrained plasma zone containing the objects or equipment to be sterilized.

### BRIEF DESCRIPTION OF THE DRAWINGS

FIG. 1A is a simplified schematic diagram of an example plasmaclave device in accordance with some embodiments.

FIG. 1B is a simplified schematic diagram of an example plasmaclave device in accordance with some embodiments.

FIG. 2 is a simplified block diagram of the example plasmaclave device shown in FIG. 1A or 1B in accordance with some embodiments.

FIG. 3 is a simplified schematic diagram of another example plasmaclave device in accordance with some embodiments.

FIG. 4 is a simplified block diagram of the example plasmaclave device shown in FIG. 3 in accordance with some embodiments.

FIG. 5 is a simplified schematic diagram of yet another example plasmaclave device in accordance with some embodiments.

FIG. 6 is a simplified block diagram of the example plasmaclave device shown in FIG. 5 in accordance with some embodiments.

FIG. 7 is a simplified schematic of a raster scanning method.

FIG. 8 is a simplified schematic of a saw-tooth scanning method.

FIG. 9 is a simplified schematic of a serpentine scanning method.

FIG. 10 is a simplified schematic of a rotating scanning method.

FIG. 11 is a simplified schematic of a scanning method over a portion of the target surface.

### DETAILED DESCRIPTION

The plasmaclave devices described herein address the shortcomings of the typical autoclave or plasma sterilization equipment by providing a solution for sterilizing a set of objects using a plasma, where the plasma spot is smaller than the object to be sterilized. In the equipment and processes described herein the plasma spot (or line) can be scanned across a target area containing objects to be sterilized using magnetic and or electric fields. The plasma can be generated in a vacuum and be higher energy than typical plasma torches. The plasma can also be pulsed. By pulsing the plasma, and scanning it across the target area, the maximum temperature of the objects can be controlled while ensuring that the object is sufficiently sterilized.

In some embodiments, a plasmaclave device used to sterilize objects comprises a chamber, and a plasma within the chamber, and the plasma is directed onto the objects in order to sterilize them. In some embodiments, the plasma is pulsed. Using a pulsed plasma is beneficial because the objects being sterilized will not be exposed to the plasma constantly over time, and in between pulses the objects will have time to cool down. In some embodiments, the plasma is spatially scanned across the objects. Using a scanned plasma is beneficial because the whole object will not be exposed to the plasma constantly over time, and will therefore not heat up as much as if the plasma were not scanned. In plasmaclave devices with a scanned plasma, a first portion of the objects being sterilized will be exposed to the plasma, and then the plasma will move, and as the plasma is directed to other portions of the object, the first portion of the object will have time to cool down. The result of using a pulsed and/or scanned plasma for sterilization of an object is that the object will be sufficiently sterilized by the plasma, but the object will not heat up as much as in typical sterilization techniques.

In some embodiments, the maximum temperature of the objects being sterilized by the plasmaclave devices described herein is less than or equal to 200° C., or less than or equal to 150° C., or less than or equal to 125° C., or less than or equal to 100° C., or from 50° C. to 200° C., or from 50° C. to 150° C., or from 50° C. to 125° C., or from 50° C. to 100° C., or within any range between a maximum temperature of 200° C., 175° C., 150° C., 125° C. or 100° C. and a minimum temperature of 50° C.

In some embodiments, the plasmaclave devices described herein contain a chamber capable of maintaining a vacuum. The pressure inside the chamber can be less than or equal to 10 Torr, or less than or equal to 1 Torr, or less than or equal to 0.1 Torr, or from 0.01 Torr to 10 Torr, or from 0.1 Torr to 10 Torr, or from 0.01 Torr to 1 Torr, or from 0.1 Torr to 1 Torr.

In some embodiments, the plasmaclave devices described herein contain a tray within the chamber, and objects to be sterilized are arranged on a target surface of the tray. In some embodiments, the tray within the chamber of the plasmaclave is stationary, or can move. The benefit of a stationary tray is that the sterilization equipment has fewer mechanical parts, which are prone to failure. The use of a scanned plasma with a stationary tray allows for the plasma to be uniformly distributed over the target area of the tray, without a movable tray requiring mechanical parts. In some embodiments, the tray is made from metal (e.g., aluminum or steel), ceramic, glass, plastic, or other appropriate material, depending on the implementation. The tray can be electrically floating, grounded, or biased, in different applications.

Plasmas can be generated in a number of ways. It should be appreciated that some of the embodiments described herein can be compatible with different methods of producing plasmas. The plasmaclave devices described herein contain one or more high voltage electrodes disposed within the chamber to generate the plasma. Alternatively, the plasma can be an inductively induced plasma.

In some embodiments, the plasmaclave devices described herein contain a plasma that is either a DC plasma or an AC plasma. The AC plasma can have a fundamental frequency from a minimum of 1 or 10 kHz to a maximum of 1 or 1.6 MHz. Additionally, the DC plasma or the AC plasma can be pulsed in some embodiments. The pulsed plasmas described herein can have a cycle repetition frequency from 1 Hz to 1 kHz with a duty cycle or pulse width between 10% and 90%. In some embodiments, the plasmaclave contains a power supply that produces the plasma and has a power level from 10 W to 100 W per 20 square inches of chamber volume.

In some embodiments, the spatial distribution of the electrical field within the chamber is influenced by fixed magnets and/or steering coils disposed within the chamber. The fixed magnets are generally used to create magnetic field spatial distributions which are substantially unchanging over time, as described below. The steering coils are generally used to create electric and magnetic field spatial distributions which change over time.

In some embodiments, the fixed magnets constrain the plasma within a constrained plasma zone within the chamber. In some embodiments, the constrained plasma zone is adjacent to (or surrounding, or in close proximity to) the target surface of the tray. In some embodiments, the fixed magnets are made of any appropriate magnetic material, such as ferrous materials, rare earth materials, etc. In some embodiments, the fixed magnets are spaced sufficiently to provide a proper constrained plasma zone or field, depending on the size of the chamber.

In some embodiments, the steering coils produce electrical and magnetic fields which focus the electrical field to a smaller region within the chamber, thereby creating a further constrained plasma within that smaller region. Additionally, the steering coils are capable of producing electric and magnetic field distributions which change over time, so they can be used to scan the further constrained plasma over the target surface of the stationary tray. In some embodiments, the steering coils are made of any appropriate material, such as magnetic wire, high temperature magnetic wire, litz wire, etc., depending on the implementation. In some embodiments, the steering coils are spaced sufficiently to provide a proper further constrained plasma zone or field, depending on the size of the chamber.

The steering coils can employ different techniques for spatially scanning the further constrained plasma across the target surface of the tray. For example, the spatial scanning can be done using a raster pattern method, or a saw-tooth pattern method, or a serpentine pattern method. In the raster, serpentine or saw-tooth scanning methods, a number of scanning rows can be defined over the target area as the number of times the scanning process begins at the first side of the target area. In any spatial scanning method, the scanning rate can be constant over the duration of the scan, or can change. Furthermore, the total number of scans over the object during sterilization (including multiple passes over the same regions) can be changed. The spatial scanning parameters (e.g., the number of scanning rows, the scanning rates over the target area, and the total number of scans over the object during sterilization) can be changed such that the duration of exposure to the plasma during a scan can be changed. The spatial scanning parameters during a scan will in turn influence the degree of sterilization and maximum temperature of the object during sterilization. The scanning parameters are therefore tuned to ensure that the object is sufficiently sterilized and the maximum temperature of the object is kept below a threshold.

In some embodiments, the objects or equipment to be sterilized are located in a portion of the target surface. The further constrained plasma is, thus, scanned over the portion of the target surface containing the objects or equipment to be sterilized.

In another embodiment, the high voltage electrodes used to generate the plasma are physically moved to control the distribution of the electric field within the chamber. Additionally, the electric field can be focused in certain areas as the electrodes move, which will further constrain the plasma in those areas. The movement of the electrodes can in turn cause the further constrained plasma to be scanned over the target area, or a portion of the target area. In some embodiments, the electrodes are moved by rotating the electrodes using a motor. In some embodiments, the electrodes are moved in different lateral directions, for example by using a gantry system. In some embodiments, the electrodes are moved back and forth above the target surface to uniformly cover the area containing in the objects to be sterilized with the further constrained plasma.

In some embodiments, a temperature sensor is included, which monitors the temperature of the objects being sterilized. The temperature sensor can be used as feedback to control the plasma parameters (e.g., the power, the frequency, the pulse parameters), or the scanning parameters. This is advantageous, because the active feedback will ensure that the maximum temperature of the object is kept below a predetermined threshold.

### Plasmaclave Device Embodiments

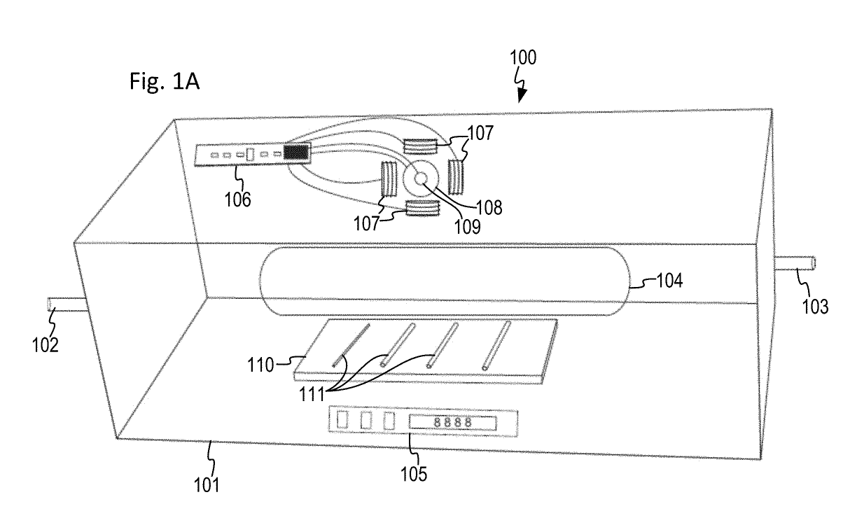

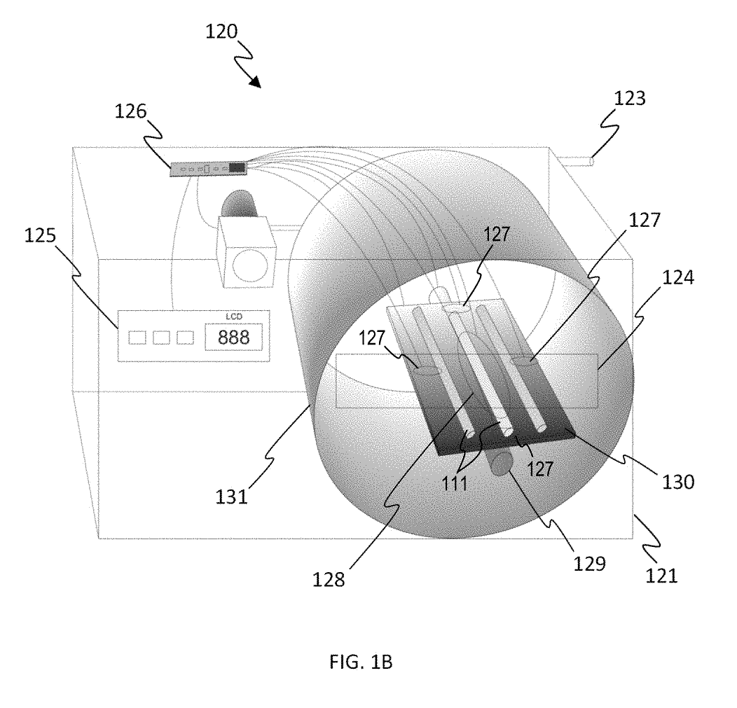

FIGS. 1A, 1B, and 2-6 show example low-cost, small-size plasmaclave devices 100, 300 and 500 in accordance with some embodiments. The plasmaclave devices 100, 300 and 500 use a plasma to sterilize any objects or equipment (e.g., medical instruments, surgical devices, food preparation utensils, tattoo devices, etc.) placed inside the plasmaclave devices 100, 300 and 500. The temperature to which the objects are heated during the plasmaclave process using these devices is considerably lower than that which occurs during an autoclave process, yet the objects are nevertheless properly sterilized. Usage of the plasmaclave devices 100, 300 and 500, thus, presents considerably less potential hazards for the operators of the devices 100, 300 and 500.

As shown in FIG. 1A, the example plasmaclave device 100 generally includes a housing 101, a power input 102, an inert gas input 103, a door 104, a control/display panel 105, a control circuit 106, steering coils (e.g., magnetic field coils) 107, a fixed magnet (e.g., a shutter field coil) 108, a high voltage electrode 109, and a tray table (i.e., tray) 110, among other components not shown for simplicity. The door 104 provides access to the interior of the housing 101. The “interior of the housing” is also referred to herein as the “chamber”, or the “plasma glow chamber”.

The geometry of the electrodes and other elements disposed within the chamber can vary in different embodiments. In some embodiments, as shown in FIG. 1B, a plasmaclave device 120 generally includes a housing 121, an inert gas input 123, a door 124, a control/display panel 125, a control circuit 126, steering coils (e.g., magnetic field coils) 127, a fixed magnet (e.g., a shutter field coil) 128, a high voltage anode electrode 129, a tray table (i.e., tray) 130, and a cylindrical cathode ground return electrode 131 (also the “chamber”), among other components not shown for simplicity. The high voltage anode electrode 129 is located within and concentric with the cylindrical cathode ground return electrode 131. For example, as shown in FIG. 1B, the high voltage anode electrode 129 can be a linear electrode surrounded by the cylindrical cathode ground return electrode 131, which serves as the chamber in this embodiment. In such embodiments, and as shown in FIG. 1B, the tray 130 can be located in the space between the high voltage anode electrode 129 and the cylindrical cathode ground return electrode 131. The figure also shows that the fixed magnet 128 and steering coils 127 can be located in positions to affect the electric and magnetic fields and constrain the plasma to a region within the target surface on the tray 130 where the objects 111 are placed. For example, the fixed magnets 128 and/or steering coils 127 could be located on either side of the high voltage anode electrode 129 and between the high voltage anode electrode 129 and the tray 130.

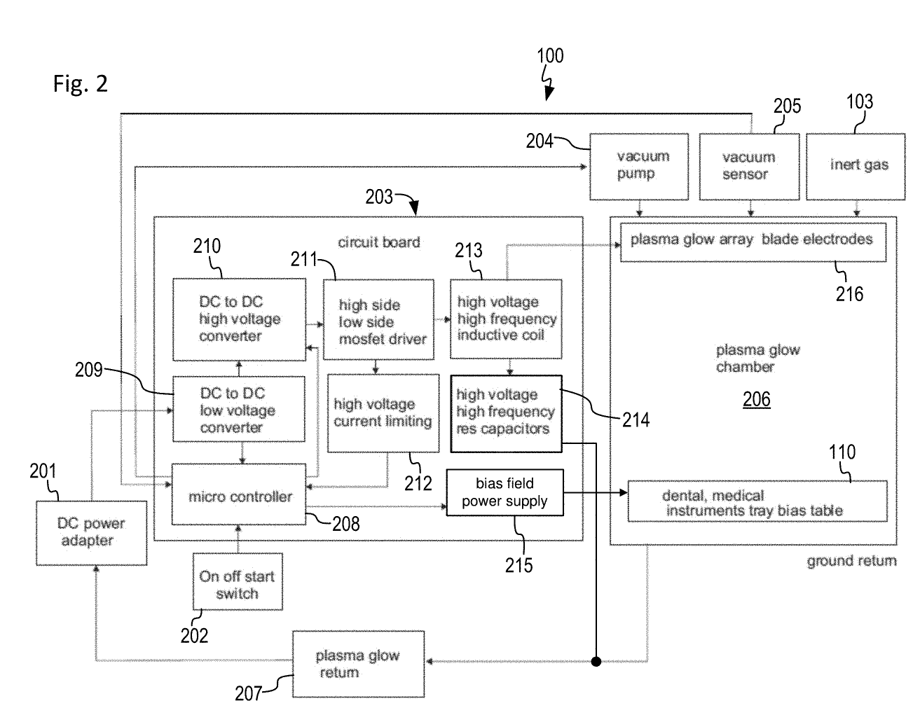

As further shown in FIG. 2, the example plasmaclave device 100 (or 120) further includes a DC power adapter 201, an on/off start switch 202, a circuit board 203, a vacuum pump 204, a pressure sensor 205, the inert gas input 103 (or 123), a plasma glow chamber 206 (i.e., the chamber), and a plasma glow ground return 207, among other components not shown for simplicity. The circuit board 203 (i.e., the control circuit 106 or 126) further includes a microcontroller 208, a DC to DC low voltage converter 209, a DC to DC high voltage converter 210, a high side/low side MOSFET driver 211, a high voltage current limiting element 212, a high voltage/high frequency inductive coil 213, high voltage/high frequency resonance capacitors 214, and a bias field power supply 215, among other components not shown for simplicity. The plasma glow chamber 206 generally contains one or more plasma glow blade electrodes 216 (i.e., the high voltage electrode 109 or 129) and the tray 110 (or 130) therein, among other components not shown for simplicity. The tray 110 (or 130) is used to hold the objects 111 for sterilization. The tray 110 (or 130) can be electrically floating, grounded, or biased, in different applications.

The power input 102 (not shown in FIG. 2) provides electrical power (e.g., AC power) to the DC power adapter 201, which powers the electrical components (e.g., the control/display panel 105 or 125, the control circuit 106 or 126, the steering coils 107 or 127, the fixed magnet 108 or 128, the high voltage electrode 109 or 129, the circuit board 203, the vacuum pump 204, and the pressure sensor 205, etc.). The inert gas input 103 (or 123) enables an inert gas to flow into the interior of the housing 101 (or 121) for the plasma to be generated in the inert gas within the plasma glow chamber 206.

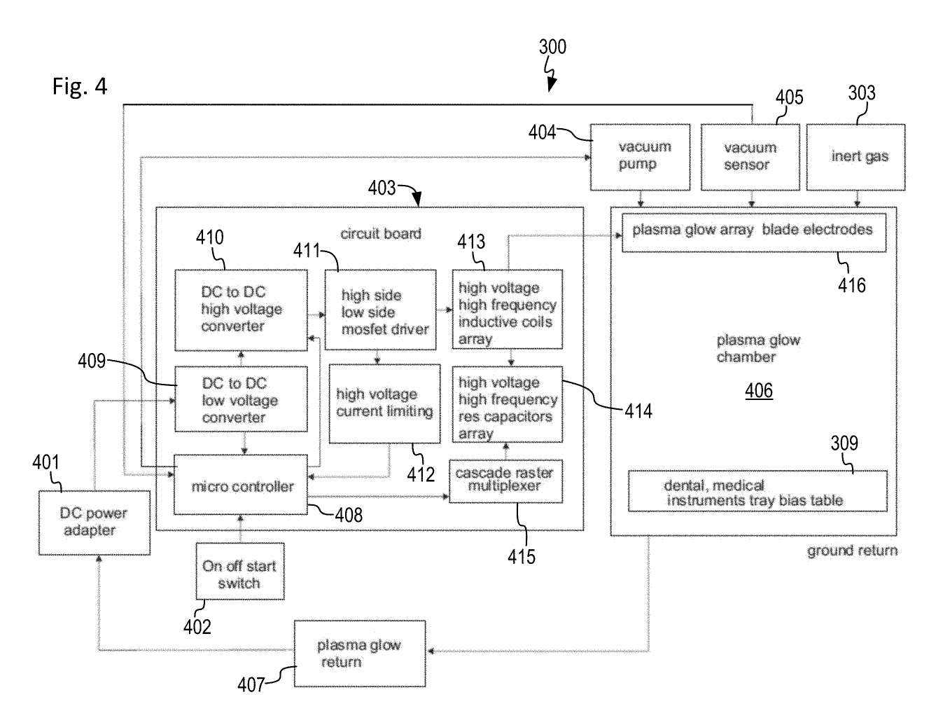

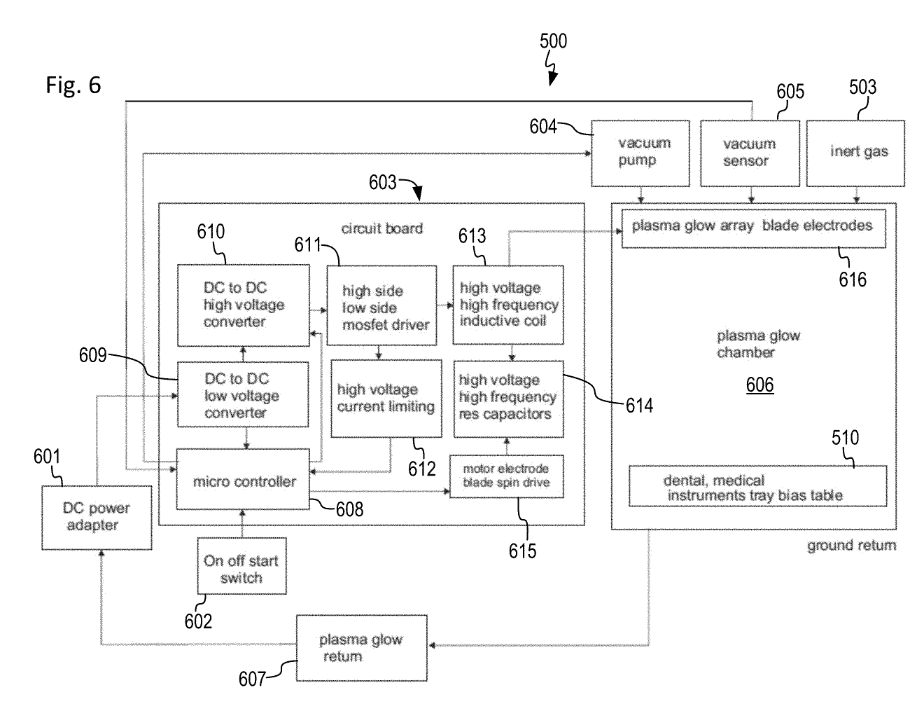

The DC power adapter 201 receives AC input power from the power input 102 and provides electrical power to the DC to DC low voltage converter 209. The DC to DC low voltage converter 209 converts the electrical power and provides the necessary current at an appropriate voltage level to the microcontroller 208 and to the DC to DC high voltage converter 210. The microcontroller 208 receives an on/off signal from the on/off start switch 202 to control when to turn on other components. The microcontroller 208 provides control signals to the DC to DC high voltage converter 210, the vacuum pump 204, and the bias field power supply 215, among other components. The DC to DC high voltage converter 210 (powered by the DC to DC low voltage converter 209, and under control of the microcontroller 208) provides a high voltage level to the high side/low side MOSFET driver 211, which provides electrical power to the high voltage/high frequency inductive coil 213. The high side/low side MOSFET driver 211 also provides a signal to the high voltage current limiting element 212, which provides feedback to the microcontroller 208 for controlling the DC to DC high voltage converter 210. The high voltage/high frequency inductive coil 213 provides electrical power to the plasma glow array blade electrodes 216 and the high voltage/high frequency resonance capacitors 214. The high voltage/high frequency resonance capacitors 214, in conjunction with the high voltage/high frequency inductive coil 213, create an LC resonant tank to generate the resonance for the high voltage applied to the plasma glow array blade electrodes 216. The bias field power supply 215 provides a bias potential to the tray 110. (Although not shown in FIGS. 4 and 6, a similar bias field power supply may provide a bias potential to the trays 309 and 510 described below.)

To perform a plasmaclave treatment on various objects 111, the door 104 (or 124) is opened by an operator of the plasmaclave device 100 (or 120), and the objects 111 are inserted into the interior of the housing 101 (or 121) and placed onto the tray table 110 (or 130). The door 104 (or 124) is then securely closed. The control/display panel 105 (or 125) (e.g., control buttons, an LCD display, etc.) is used to set a time period for performing, and/or to start and stop, the plasmaclave treatment. The control panel/display panel 105 (or 125) is also used to set other parameters (e.g., scanning parameters, plasma power parameters, inert gas flow, pressure, etc.) for the process in different embodiments.

When the plasmaclave treatment is started, the air inside the housing 101 (or 121) is pumped out using the vacuum pump 204 (under control of the microcontroller 208) until the pressure sensor 205 indicates that the pressure in the plasma glow chamber 206 is sufficiently low. The pressure sensor 205 can be used as a safety interlock control input, where the process is not continued (e.g., the inert gas flow is not started, the high voltage is not delivered to the blade electrodes, etc.) until a sufficiently low pressure is reached within the chamber. Once the pressure is sufficiently low, then the inert gas is flowed into the interior of the housing (i.e., the chamber) 101 (or 121). Then the plasma glow array blade electrodes 216 receive power, as described above to generate a plasma in the inert gas in the chamber. In some embodiments, the plasma is generated in a plasma region or zone generally between the high voltage electrode 109 (or 129) and the tray table 110 (or 130). In some embodiments, the plasma glow chamber 206 is grounded through the plasma glow ground return 207 to complete the circuit to the DC power adapter 201 and to help direct the plasma. The fixed magnet 108 (or 128) controls and intensifies the electric field, which in turn creates a higher density plasma in a constrained plasma zone within the chamber. FIG. 1A (or 1B) shows the fixed magnet 108 (or 128) arranged adjacent to the high voltage electrodes 109 (or 129). However, in different embodiments, other fixed magnets can be distributed in other locations around the chamber such that the electric field is focused and the plasma is constrained in a selected constrained plasma zone. In some embodiments, the constrained plasma zone is located adjacent to the tray 110 (or 130), where the objects 111 to be sterilized are located. The steering coils 107 (or 127) are operated to further control where the electric field is focused and further constrain the plasma. The further constrained plasma can then be scanned over a target surface over the tray table 110 (or 130), thereby sterilizing the objects 111. In some embodiments, the further constrained plasma is scanned throughout the extent of the tray table 110 (or 130), while in other embodiments, the further constrained plasma is scanned over a portion of the tray table 110 (or 130).

When the plasmaclave treatment is finished, the power to the steering coils 107 (or 127), the fixed magnet 108 (or 128), and the high voltage electrode 109 (or 129) is turned off. Then the inert gas is optionally flushed from the interior of the housing 101 (or 121). After the chamber is vented to atmosphere, the door 104 (or 124) can be opened and the objects 111 removed.

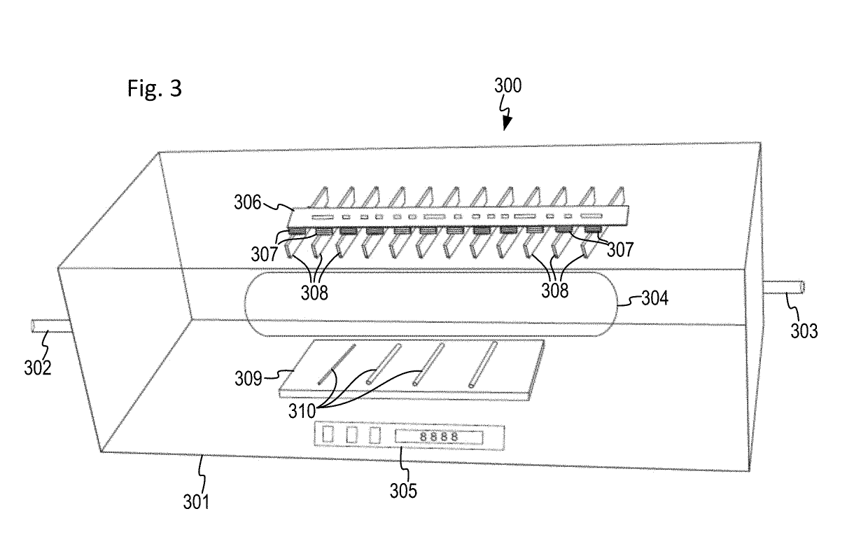

As shown in FIG. 3, the example plasmaclave device 300 generally includes a housing 301, a power input 302, an inert gas input 303, a door 304, a control/display panel 305, a control circuit 306, a series of steering coils (e.g., high voltage cascade raster coils) 307, a series of high voltage electrodes (e.g., blade electrodes) 308, and a tray 309, among other components not shown for simplicity. The door 304 provides access to the interior of the housing 301, i.e., the chamber, the plasma chamber, or the plasma glow chamber. As further shown in FIG. 4, the example plasmaclave device 300 further includes a DC power adapter 401, an on/off start switch 402, a circuit board 403, a vacuum pump 404, a pressure sensor 405, the inert gas input 303, a plasma glow chamber 406, and a plasma glow ground return 407, among other components not shown for simplicity. The circuit board 403 (i.e., the control circuit 306) further includes a microcontroller 408, a DC to DC low voltage converter 409, a DC to DC high voltage converter 410, a high side/low side MOSFET driver 411, a high voltage current limiting element 412, a high voltage/high frequency inductive coils array 413, a high voltage/high frequency resonance capacitors array 414, and a cascade raster multiplexer 415, among other components not shown for simplicity. The plasma glow chamber 406 generally contains plasma glow array blade electrodes 416 (i.e., the high voltage electrodes 308) and the tray 309 therein, among other components not shown for simplicity. The tray 309 is used to hold the objects for sterilization. The tray 309 can be electrically floating, grounded, or biased, in different applications.

The power input 302 provides electrical power (e.g., AC power) to the DC power adapter 401, which powers the electrical components (e.g., the control/display panel 305, the control circuit 306, the steering coils 307, high voltage electrodes 308, the circuit board 403, the vacuum pump 404, and the pressure sensor 405, etc.). The inert gas input 303 enables an inert gas to flow into the interior of the housing 301 for the plasma to be generated in the inert gas within the plasma glow chamber (i.e., the chamber) 406.

The DC power adapter 401 receives AC input power from the power input 302 (not shown in FIG. 4) and provides electrical power to the DC to DC low voltage converter 409. The DC to DC low voltage converter 409 converts the electrical power and provides the necessary current at an appropriate voltage level to the microcontroller 408 and to the DC to DC high voltage converter 410. The microcontroller 408 receives an on/off signal from the on/off start switch 402 to control when to turn on other components. The microcontroller 408 provides control signals to the DC to DC high voltage converter 410, the vacuum pump 404, and the cascade raster multiplexer 415, among other components. The DC to DC high voltage converter 410 (powered by the DC to DC low voltage converter 409, and under control of the microcontroller 408) provides a high voltage level to the high side/low side MOSFET driver 411, which provides electrical power to the high voltage/high frequency inductive coils array 413. The high side/low side MOSFET driver 411 also provides a signal to the high voltage current limiting element 412, which provides feedback to the microcontroller 408 for controlling the DC to DC high voltage converter 410. The high voltage/high frequency inductive coils array 413 provides electrical power to the plasma glow array blade electrodes 416 and the high voltage/high frequency resonance capacitors array 414. The high voltage/high frequency resonance capacitors array 414 also receive control signals from the cascade raster multiplexer 415. The high voltage/high frequency resonance capacitors array 414, in conjunction with the high voltage/high frequency inductive coils array 413, create an LC resonant tank to generate the resonance for the high voltage applied to the plasma glow array blade electrodes 416.

To perform a plasmaclave treatment on various medical instruments 310, the door 304 is opened by an operator of the plasmaclave device 300, and the medical instruments 310 are inserted into the interior of the housing 301 and placed onto the tray table 309. The door 304 is then securely closed. The control/display panel 305 (e.g., control buttons, an LCD display, etc.) is used to set a time period for performing, and/or to start and stop, the plasmaclave treatment. The control panel/display panel 305 is also used to set other parameters (e.g., scanning parameters, plasma power parameters, inert gas flow, pressure, etc.) for the process in different embodiments.

When the plasmaclave treatment is started, the air inside the housing 301 is pumped out using the vacuum pump 404 (under control of the microcontroller 408) until the pressure sensor 405 indicates that the pressure in the plasma glow chamber 406 is sufficiently low. The pressure sensor 405 can be used as a safety interlock control input, where the process is not continued (e.g., the inert gas flow is not started, the high voltage is not delivered to the blade electrodes, etc.) until a sufficiently low pressure is reached within the chamber. Once the pressure is sufficiently low, then the inert gas is flowed into the interior of the housing (i.e., the chamber) 301. Then the plasma glow array blade electrodes 416 receive power, as described above to generate a plasma in the inert gas in the chamber. In some embodiments, the plasma is generated in a plasma region or zone generally between the high voltage electrodes 308 and the tray table 309. In some embodiments, the plasma glow chamber 406 is grounded through the plasma glow ground return 407 to complete the circuit to the DC power adapter 401 and to help direct the plasma. In some embodiments, one or more fixed magnets (not shown in FIG. 3) control and intensify the electric field, which in turn creates a higher density plasma in a constrained plasma zone within the chamber. These fixed magnets are shutter field coils surrounding the high voltage electrodes 308, in some embodiments. In other embodiments, other fixed magnets can be distributed in other locations around the chamber such that the electric field is focused and the plasma is constrained in a selected constrained plasma zone. In some embodiments, the constrained plasma zone is located adjacent to the tray, where the objects to be sterilized are located. The steering coils 307 are operated to further control where the electric field is focused and further constrain the plasma. The further constrained plasma can then be scanned over a target surface over the tray table 309, thereby sterilizing the objects 310. In some embodiments, the electrical signals from the control circuit 306 are applied in a rapid raster scan pattern to the high steering coils 307, so that the further constrained plasma is rapidly scanned over a target surface of the tray table 309, thereby sterilizing the medical instruments 310. In some embodiments, the further constrained plasma is scanned throughout the extent of the tray table, while in other embodiments, the further constrained plasma is scanned over a portion of the tray table.

When the plasmaclave treatment is finished, the power to the steering coils 307 and the high voltage electrodes 308 is turned off. Then the inert gas is optionally flushed from the interior of the housing 301. After the chamber is vented to atmosphere, the door 304 can then be opened and the objects 310 removed.

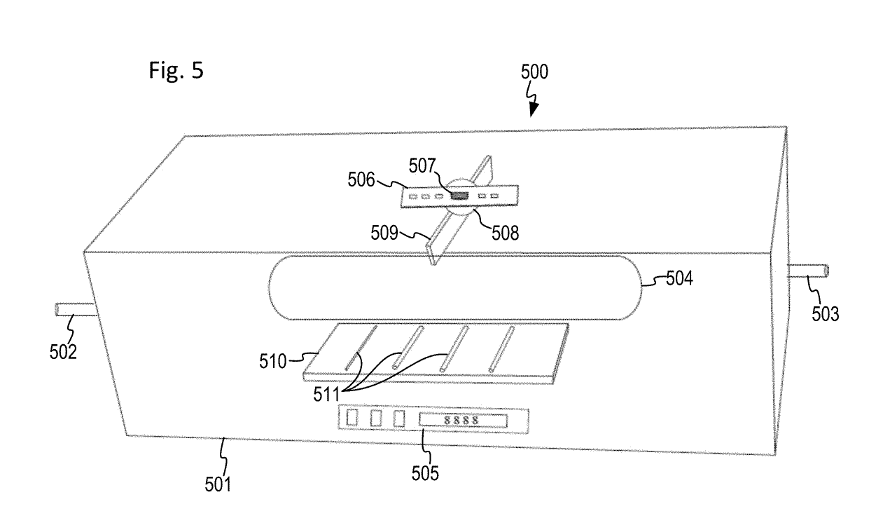

As shown in FIG. 5, the example plasmaclave device 500 generally includes a housing 501, a power input 502, an inert gas input 503, a door 504, a control/display panel 505, a control circuit 506, a high voltage coil 507, a motor 508, a high voltage electrode (e.g., a blade electrode) 509, and a tray table 510, among other components not shown for simplicity. The door 504 provides access to the interior of the housing 501, i.e., the chamber, the plasma chamber, or the plasma glow chamber. As further shown in FIG. 6, the example plasmaclave device 500 further includes a DC power adapter 601, an on/off start switch 602, a circuit board 603, a vacuum pump 604, a pressure sensor 605, the inert gas input 503, a plasma glow chamber (i.e., the chamber) 606, and a plasma glow ground return 607, among other components not shown for simplicity. The circuit board 603 (i.e., the control circuit 506) further includes a microcontroller 608, a DC to DC low voltage converter 609, a DC to DC high voltage converter 610, a high side/low side MOSFET driver 611, a high voltage current limiting element 612, a high voltage/high frequency inductive coil 613, high voltage/high frequency resonance capacitors 614, and a motor electrode blade spin drive 615, among other components not shown for simplicity. The plasma glow chamber (i.e., the chamber) 606 generally contains plasma glow array blade electrodes 616 (i.e., the high voltage electrode 509) and the tray table (i.e., the tray) 510 therein, among other components not shown for simplicity. The tray 510 is used to hold the objects (e.g., dental, medical instruments) for sterilization. The tray can be electrically floating, grounded, or biased, in different applications.

The power input 502 provides electrical power (e.g., AC power) to the DC power adapter 601, which powers the electrical components (e.g., the control/display panel 505, the control circuit 506, the high voltage coil 507, the motor 508, the high voltage electrode 509, the circuit board 603, the vacuum pump 604, and the pressure sensor 605, etc.). The inert gas input 503 enables an inert gas to flow into the interior of the housing (i.e., the chamber) 501 for the plasma to be generated in the inert gas within the plasma glow chamber (i.e., the chamber) 606.

The DC power adapter 601 receives AC input power from the power input 502 and provides electrical power to the DC to DC low voltage converter 609. The DC to DC low voltage converter 609 converts the electrical power and provides the necessary current at an appropriate voltage level to the microcontroller 608 and to the DC to DC high voltage converter 610. The microcontroller 608 receives an on/off signal from the on/off start switch 602 to control when to turn on other components. The microcontroller 608 provides control signals to the DC to DC high voltage converter 610, the vacuum pump 604, and the motor electrode blade spin drive 615, among other components. The DC to DC high voltage converter 610 (powered by the DC to DC low voltage converter 609, and under control of the microcontroller 608) provides a high voltage level to the high side/low side MOSFET driver 611, which provides electrical power to the high voltage/high frequency inductive coil 613. The high side/low side MOSFET driver 611 also provides a signal to the high voltage current limiting element 612, which provides feedback to the microcontroller 608 for controlling the DC to DC high voltage converter 610. The high voltage/high frequency inductive coil 613 provides electrical power to the plasma glow array blade electrodes 616 and the high voltage/high frequency resonance capacitors 614. The high voltage/high frequency resonance capacitors 614 also receive control signals from the motor electrode blade spin drive 615.

To perform a plasmaclave treatment on various objects 511, the door 504 is opened by an operator of the plasmaclave device 500, and the objects 511 are inserted into the interior of the housing (i.e., chamber) 501 and placed onto the tray table (i.e., tray) 510. The door 504 is then securely closed. The control/display panel 505 (e.g., control buttons, an LCD display, etc.) is used to set a time period for performing, and/or to start and stop, the plasmaclave treatment. The control panel/display panel 505 is also used to set other parameters (e.g., scanning parameters, plasma power parameters, inert gas flow, pressure, etc.) for the process in different embodiments.

When the plasmaclave treatment is started, the air inside the housing 501 is pumped out using the vacuum pump 604 (under control of the microcontroller 608) until the pressure sensor 605 indicates that the pressure in the plasma glow chamber 606 is sufficiently low. The pressure sensor 605 can be used as a safety interlock control input, where the process is not continued (e.g., the inert gas flow is not started, the high voltage is not delivered to the blade electrodes, etc.) until a sufficiently low pressure is reached within the chamber. Once the pressure is sufficiently low, then the inert gas is flowed into the interior of the housing (i.e., the chamber) 501. Then the plasma glow array blade electrodes 616 receive power, as described above to generate a plasma in the inert gas in the chamber. In some embodiments, the plasma is generated in a plasma region or zone generally between the high voltage electrodes 509 and the tray table 510. In some embodiments, the plasma glow chamber 606 is grounded through the plasma glow ground return 607 to complete the circuit to the DC power adapter 601. In some embodiments, one or more fixed magnets (not shown in FIG. 5) control and intensify the electric field, which in turn creates a higher density plasma in a constrained plasma zone within the chamber. These fixed magnets are shutter field coils surrounding the high voltage electrodes, in some embodiments. In other embodiments, other fixed magnets can be distributed in other locations around the chamber such that the electric field is focused and the plasma is constrained in a selected constrained plasma zone. In some embodiments, the constrained plasma zone is located adjacent to the tray, where the objects to be sterilized are located. The motor 508 is operated to rotate the high voltage electrode, thereby further controlling where the electric field is distributed and further constraining the plasma. As the motor rotates the high voltage electrodes, the further constrained plasma is scanned over the target surface over the tray table 510, thereby sterilizing the objects 511.

When the plasmaclave treatment is finished, the power to the high voltage coil 507, the motor 508, and the blade electrode 509 is turned off. Then the inert gas is optionally flushed from the interior of the housing 501. After the chamber is vented to atmosphere, the door 504 can then be opened and the medical instruments 511 removed.

Plasmaclave Device Scanning Methods

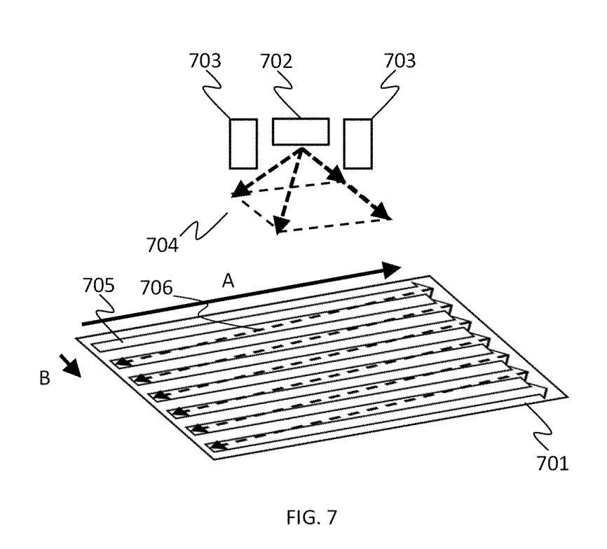

FIG. 7 shows a rastering method for scanning the plasma over a target surface 701. In this rastering method, an electric field is generated by a high voltage electrode 702, which generates a plasma. Steering coils 703 create additional electric and magnetic field spatial distributions which change over time as illustrated by dashed arrows 704, resulting in a higher density plasma focused in a spot which changes location over time. The size of this “plasma spot” can vary from nearly the same size as the tray to very small (e.g., 0.01 times the area of the tray, or 0.001 times the area of the tray). In the rastering method, the steering coils first scan the plasma spot in a direction from a first side of the target surface to a second side of the target surface 701 (as indicated by large arrows 705) at a first scanning rate, and then return (as indicated by dashed arrows 706) to the first side of the target surface at a second scanning rate which is faster than the first scanning rate, before starting the sequence again. A time period for completing a scan cycle may be between 0.1 second and 1 second per cycle.

FIG. 7 shows a first motion (at first large arrow 705) of the plasma spot in a direction along vector A, from a first side of the target surface 701 to a second side of the target surface 701. Then the plasma spot is quickly scanned (along dashed arrow 706) back to the first side of the target surface 701 to start a second row (at second large arrow 705) that is translated in the direction B. By stepping each scan in the direction B, the whole target surface will eventually be covered after a certain number of scans. This number of scans can be 5, 10, 20, 50, 100, 500, 1000, or from 5 to 1000, or from 5 to 500, or from 5 to 100, or from 5 to 50, or from 5 to 20. Generally, the magnitude of the vector B in FIG. 7 (i.e., the distance between scans in the B direction) is chosen such that it is equal to or less than the of the size of the plasma spot. It is understood that the plasma spot will have some peaked distribution, and the “size” of the spot can be defined by the full-width at half maximum of the peak. If the return speed from the second side of the target surface 701 to the first side of the target surface 701 is much faster than the first scanning speed, then the rastering method is relatively efficient.

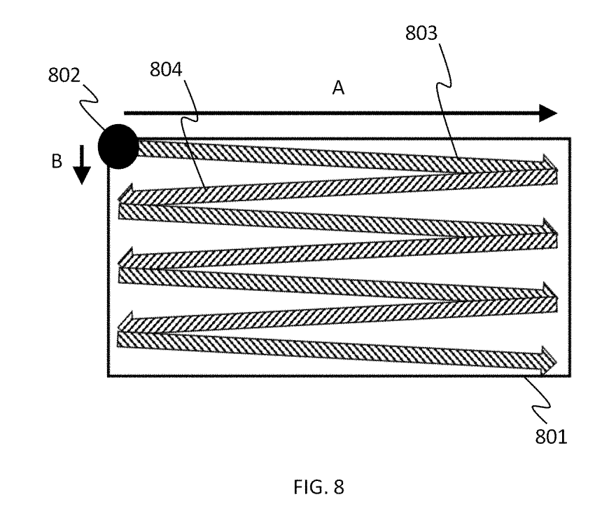

FIG. 8 shows a saw-tooth method for scanning the plasma over a target surface 801. Similar to the rastering method described above, an electric field is generated by a high voltage electrode, which generates a plasma. Steering coils create additional electric and magnetic field spatial distributions which change over time, resulting in a higher density plasma focused in a spot which changes location over time. The size of this “plasma spot” can vary from nearly the same size as the tray to very small (e.g., 0.01 times the area of the tray, or 0.001 times the area of the tray). In the saw-tooth method, the steering coils scan the plasma spot in a direction from a first side to a second side of the target surface 801 at a slight angle (e.g., as illustrated by large arrow 803) and at a first scanning rate, and then scan from the second side back to the first side at a slight angle (e.g., as illustrated by large arrow 804) and at the first scanning rate, before starting the scanning sequence again. The slight angles can be chosen to cover the whole target surface with a certain number of scans. A time period for completing a scan cycle may be between 0.1 second and 1 second per cycle.

FIG. 8 shows a first motion of the plasma spot (in the direction of arrow 803) in a diagonal direction along the sum of vector A and vector B, from a first side of the target surface 801 to a second side of the target surface 801. Then the plasma spot is scanned back to the first side of the target surface 801 in a diagonal direction (arrow 804) along the sum of the negative of the vector A and vector B. Each scan ends farther down the target surface in the direction B, and the whole target surface will eventually be covered after a certain number of scans. This number of scans can be 5, 10, 20, 50, 100, 500, 1000, or from 5 to 1000, or from 5 to 500, or from 5 to 100, or from 5 to 50, or from 5 to 20. FIG. 8 also shows the plasma spot size 802. Generally, the magnitude of the vector B in FIG. 8 (i.e., the distance between the beginning of scans in the B direction) is chosen such that it is equal to or less than the of the size of the plasma spot. It is understood that the plasma spot will have some peaked distribution, and the “size” of the spot can be defined by the full-width at half maximum of the peak. One advantage of the saw-tooth method is that there is only one scanning speed needed. One disadvantage is that the angled scans create target areas that are covered by the plasma spot more often and regions that are covered by the plasma spot less often.

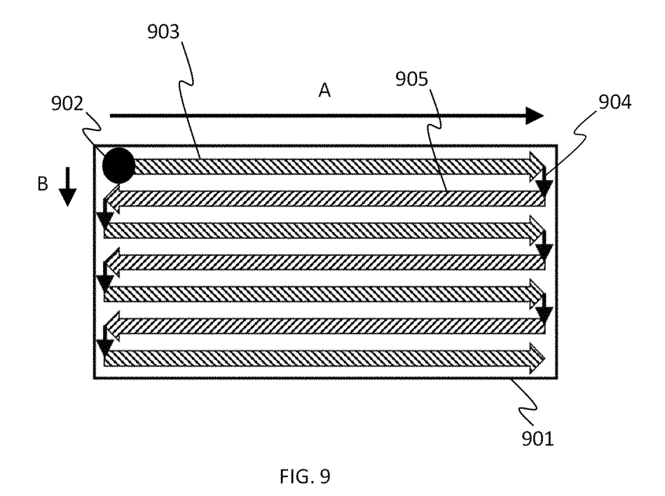

FIG. 9 shows a serpentine method for scanning the plasma over the target surface 901. Similar to the rastering method described above, an electric field is generated by a high voltage electrode, which generates a plasma. Steering coils create additional electric and magnetic field spatial distributions which change over time, resulting in a higher density plasma focused in a spot which changes location over time. The size of this “plasma spot” can vary from nearly the same size as the tray to very small (e.g., 0.01 times the area of the tray, or 0.001 times the area of the tray). In the serpentine method, steering coils scan the plasma in a direction from a first side to a second side of the target surface 901 at a first scanning rate (along arrow 903), and then step to a new row (along arrow 904), and scan from the second side of the target surface 901 back to the first side of the target surface 901 at the first scanning rate (along arrow 905), before starting the scanning sequence again. A time period for completing a scan cycle may be between 0.1 second and 1 second per cycle.

FIG. 9 shows a first motion of the plasma spot (arrow 903) in a direction along vector A, from a first side of the target surface 901 to a second side of the target surface 901. Then the plasma spot is quickly scanned (arrow 904) in the direction of the vector B to start a second row that is translated down the target area. Then the plasma is scanned (arrow 905) in the negative of the direction A. By stepping each scan in the direction B, the whole target surface will eventually be covered after a certain number of scans. This number of scans can be 5, 10, 20, 50, 100, 500, 1000, or from 5 to 1000, or from 5 to 500, or from 5 to 100, or from 5 to 50, or from 5 to 20. Generally, the magnitude of the vector B in FIG. 9 (i.e., the distance between scans in the B direction) is chosen such that it is equal to or less than the of the size of the plasma spot. It is understood that the plasma spot will have some peaked distribution, and the “size” of the spot can be defined by the full-width at half maximum of the peak. One advantage of the serpentine method is that one scanning speed can be used, and the target area will be relatively uniformly covered by the plasma spot.

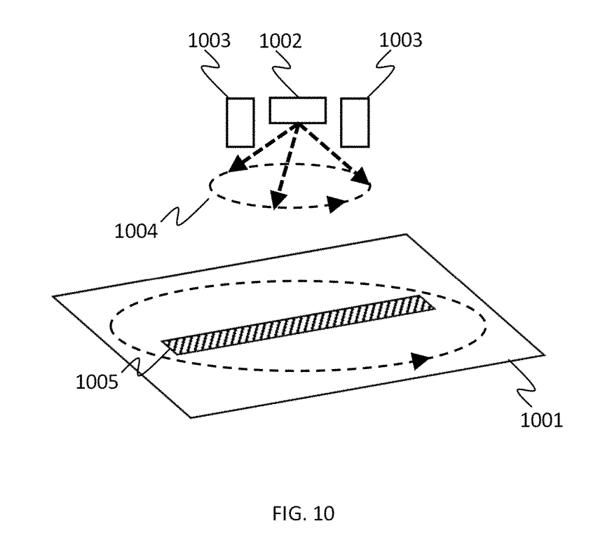

FIG. 10 shows a rotating line method for scanning the plasma over the target surface 1001. Similar to the rastering method described above, an electric field is generated by a high voltage electrode 1002, which generates a plasma. In this method, the high voltage electrode creates an electric field that is in the shape of a line. Steering coils 1003 create additional electric and magnetic field spatial distributions which change over time as illustrated by dashed arrows 1004, resulting in a higher density plasma focused in a line 1005 which changes location over time. The length of this “plasma line” 1005 is roughly the same length as the target area, and the width of the line can vary from nearly the same size as the tray to very small (e.g., 0.01 times the area of the tray, or 0.001 times the area of the tray). In the rotating line method, steering coils rotate the plasma line to cover the whole target area. Alternatively, the electrode 1002 producing a plasma line could itself be rotated (e.g., by a motor), and produce a similar rotating plasma line. One disadvantage of this method is the corners of the target area will not be covered by the plasma line. A time period for completing a scan cycle may be between 0.1 second and 1 second per cycle.

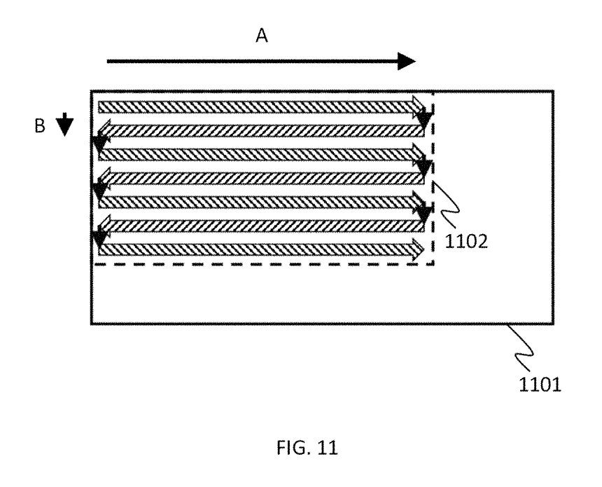

FIG. 11 shows an example of a method for scanning the plasma over the target surface 1101, where a portion 1102 of the target surface 1101 is covered by the plasma. In this example, the objects to be sterilized are all placed within the area portion 1102, and therefore it is sufficient to scan the plasma over area portion 1102, and there is no need to scan the plasma over the whole target surface 1101. FIG. 11 illustrates covering a portion of the target surface using a serpentine scan, however any scanning method could be used to cover a portion of a target surface in other embodiments. A time period for completing a scan cycle may be between 0.1 second and 1 second per cycle.

Although embodiments of the present invention have been discussed primarily with respect to specific embodiments thereof, other variations are possible. Various configurations of the described system may be used in place of, or in addition to, the configurations presented herein. For example, additional components may be included in circuits where appropriate. As another example, configurations were described with general reference to certain types and combinations of circuit or system components, but other types and/or combinations of circuit components could be used in addition to or in the place of those described.

Those skilled in the art will appreciate that the foregoing description is by way of example only, and is not intended to limit the present invention. Nothing in the disclosure should indicate that the present invention is limited to systems that have the specific type of devices shown and described. Nothing in the disclosure should indicate that the present invention is limited to systems that require a particular form of integrated circuits or hardware components, except where specified. In general, any diagrams presented are only intended to indicate one possible configuration, and many variations are possible. Those skilled in the art will also appreciate that methods and systems consistent with the present invention are suitable for use in a wide range of applications.

While the specification has been described in detail with respect to specific embodiments of the present invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention.

1. A plasmaclave device used to sterilize objects, comprising:
a chamber;
a stationary tray disposed within the chamber, the stationary tray having a target surface and being configured to hold the objects on the target surface;
a constrained plasma zone within the chamber adjacent to the target surface of the stationary tray;
one or more high voltage electrodes disposed within the chamber;
fixed magnets disposed within the chamber; and
steering coils disposed within the chamber;
wherein:
the high voltage electrodes generate an electric field that creates a plasma within the chamber;
the fixed magnets focus the electric field constraining the plasma into the constrained plasma zone of the of the chamber; and
the steering coils further constrain the plasma, and scan the further constrained plasma over the target surface of the stationary tray.

2. The plasmaclave device of claim 1, wherein the plasma is a DC plasma.

3. The plasmaclave device of claim 2, wherein the DC plasma is pulsed with a repetition frequency from 1 Hz to 1 kHz.

4. The plasmaclave device of claim 1, wherein the plasma is an AC plasma with a frequency from 1 kHz to 1 MHz.

5. The plasmaclave device of claim 4, wherein the AC plasma is pulsed with a repetition frequency from 1 Hz to 1 kHz.

6. The plasmaclave device of claim 1, wherein a pressure in the chamber is from 1 Torr to 10 Torr.

7. The plasmaclave device of claim 1, further including a power supply that produces the plasma at a power level from 10 W to 100 W per 20 square inches of chamber volume.

8. The plasmaclave device of claim 1, wherein the stationary tray comprises aluminum.

9. A method of sterilizing objects, comprising:
providing a chamber having a stationary tray and a constrained plasma zone therein, the objects being on a target surface of the stationary tray;
creating a plasma within the chamber by generating an electric field therein;
pulsing the plasma at a repetition frequency using a power supply;
constraining the plasma into the constrained plasma zone by focusing the electric field using fixed magnets; and
further constraining the plasma and scanning the further constrained plasma across the objects on the target surface of the stationary tray using steering coils.

10. The method of claim 9, wherein the plasma is a DC plasma.

11. The method of claim 9, wherein the plasma is an AC plasma with a frequency from 1 kHz to 1 MHz.

12. The method of claim 9, wherein the repetition frequency is from 1 Hz to 1 kHz.

13. The method of claim 9, wherein the chamber is at a pressure from 0.1 mTorr to 10 Torr.

14. The method of claim 9, wherein a power supplied to the plasma is from 10 W to 100 W per 20 square inches of chamber volume.

15. The method of claim 9, wherein a maximum temperature of the objects or equipment being sterilized is less than or equal to 100° C.

16. The method of claim 9, wherein the constrained plasma is scanned over the constrained plasma zone in a serpentine pattern.

17. The method of claim 9, wherein the constrained plasma is scanned over the constrained plasma zone in a raster pattern.

18. The method of claim 9, wherein a time period for completing a scan cycle is between 0.1 second and 1 second per cycle.

19. The method of claim 9, wherein the objects or equipment to be sterilized are located in a portion of the constrained plasma zone, and the further constrained plasma is scanned over the portion of the constrained plasma zone containing the objects or equipment to be sterilized.
